(19) United States
(12) Patent Application Publication
(10) Pub. No.: US 2011/0130303 A1
(43) Pub. Date: Jun. 2, 2011

(54) IN VITRO DIAGNOSIS/PROGNOSIS METHOD AND KIT FOR ASSESSMENT OF TOLERANCE IN LIVER TRANSPLANTATION

(75) Inventors: Alberto Sanchez Fueyo, Barcelona (ES); Juan José Lozano Salvatella, Barcelona (ES)

(73) Assignee: Institut D'Investigacions Biomediques August PI I Sunyer (IDIBAPS)

(21) Appl. No.: 13/000,931

(22) PCT Filed: Jul. 3, 2009
(86) PCT No.: PCT/EP09/58441

§ 371 Date: Feb. 15, 2011

(30) Foreign Application Priority Data
Jul. 3, 2008 (EP) PCT/EP2008/058592

(51) Int. Cl. C40B 30/04; G01N 33/53; C40B 30/00; C12Q 1/68; G01N 33/50; C40B 40/00; C40B 40/06; C40B 40/10; C12M 1/34

(52) U.S. Cl. 506/9; 435/7.25; 506/7; 435/6.12; 436/501; 436/86; 506/13; 506/16; 506/18; 435/287.2

(57) ABSTRACT

In vitro diagnosis/prognosis method and kit, for assessment of tolerance in liver transplantation. The present invention refers to the study of peripheral blood transcriptional patterns from 80 liver transplant recipients and 16 non-transplanted healthy individuals employing either oligonucleotide microarrays and/or quantitative real-time PCR to design a clinically applicable molecular test. This has resulted in the discovery and validation of several gene signatures comprising a modest number of genes capable of identifying tolerant and non-tolerant recipients with high accuracy. The marker genes are KLRF1, SLAMF7, NKG7, IL2RB, KLRB1, FANCG, GNPTAB, CLIC3, PSMD14, ALG8, CX3CR1, RGS 3. Multiple peripheral blood lymphocyte subsets contribute to the tolerance-associated transcriptional patterns with NK and γdelta T cells exerting a predominant influence. The invention concludes that transcriptional profiling of peripheral blood can be employed to identify liver transplant recipients who can discontinue immunosuppressive therapy and that innate immune cells are likely to play a major role in the maintenance of operational tolerance in liver transplantation.

### FIELD OF THE INVENTION

This invention refers to the field of human medicine, and specifically to the diagnosis of the tolerant state in liver transplant recipients.

### STATE OF THE ART

Maintenance of a normal allograft function despite complete discontinuation of all immunosuppressive drugs is occasionally reported in clinical organ transplantation, particularly following liver transplantation (1). Patients spontaneously accepting their grafts are conventionally considered as “operationally” tolerant, and provide a proof-of concept that immunological tolerance can actually be attained in humans. We and others have documented differences in the phenotype and gene expression of peripheral blood mononuclear cells (PBMCs) obtained from operationally tolerant liver recipients as compared with patients requiring on-going pharmacological immunosuppression (2, 4). While these observations have provided valuable information on the cellular and molecular basis of human operational tolerance, the translation of this information into a clinically applicable molecular diagnostic test capable of identifying tolerance remains a challenge.

The long-term survival of transplanted grafts critically depends on the life-long administration of immunosuppressive drugs to prevent graft rejection. These drugs are very effective at preventing graft rejection, but they are also associated with severe side effects, such as nephrotoxicity, an augmented risk of opportunistic infections and tumors, and metabolic complications such as diabetes, hyperlipidemia and arterial hypertension. Due to the side effects of immunosuppressive drugs, the induction of tolerance, defined as a state in which the graft maintains a normal function in the absence of chronic immunosuppression, is one of the main goals of research in transplant immunology. Tolerance induction is possible in a great number of experimental models of transplant in rodents. Nevertheless, the application of these experimental treatments in the clinic has been a failure to a large extent. Liver transplantation is the only clinical setting in which tolerance spontaneously occurs in a substantial proportion of patients. Indeed, complete immunosuppression withdrawal can be achieved in around 21% of patients (1). Unfortunately, there are currently no means to identify these patients before immunosuppression withdrawal is attempted. For this reason, complete discontinuation of immunosuppressive drugs is rarely attempted in liver transplantation, and thus many patients continue to be unnecessarily immunosuppressed, with the health and economic problems that this involves.

One of the reasons why clinical application in humans of experimental treatments of tolerance induction has not been successful relates to the lack of an accurate tool to non-invasively diagnose tolerance in human transplant recipients. Recent publications point out the urgent need for this tool (e.g. 19-20).

Prior attempts to identify tolerance in transplantation, mainly of kidney and liver, have employed either antigen-specific functional assays or antigen-nonspecific tests. In the functional assays recipient T lymphocytes are challenged with donor antigens either in vitro or in vivo (cf. 21-23). These assays are very valuable from a mechanistic point of view, since they are the only tests capable of revealing which pathways are responsible for the specificity of the tolerance state. Unfortunately, these assays are also difficult to perform, highly variable from laboratory to laboratory (difficult to standardize), and require the availability of carefully cryopreserved donor cells. For these reasons, functional assays are not optimal for widespread clinical application, and are currently employed only in selected, highly specialized laboratories, and basically for research purposes.

The antigen-non specific immune monitoring tests constitute a variety of methodologies aiming at the phenotypic characterization of the recipient immune system, without the use of donor antigen challenges. Among these tests, the study of T cell receptor CDR3 length distribution patterns (TcLandscape, see 24) and peripheral blood cell immunophenotyping employing flow cytometry, have been employed to identify biomarkers characteristic of tolerance in humans. The TcLandscape technique has been employed in peripheral blood to discriminate between tolerant kidney recipients and recipients experiencing chronic rejection (cf. 25). However, this technique is expensive, is currently only available at one laboratory (Inserm 643 and TcLand Expression in Nantes, France), and has never been validated in liver transplantation. The use of peripheral blood immunophenotyping has been used with peripheral blood samples from both liver and kidney tolerant transplant recipients. Two studies addressing this methodology are known to inventors. In the first one, from the University of Pittsburgh in USA (cf. 4), it is said that the ratio between pDC and mDC dendritic cell subsets could discriminate between tolerant and non-tolerant recipients in pediatric liver transplantation. In the second study, from Kyoto (cf. 2), it is said that an increased ratio between delta-1 and delta-2 gammadelta T cells in peripheral blood is more prevalent in tolerant than in non-tolerant liver recipients. However, none of these tests offers the accuracy required for the widespread clinical application.

While the chronic use of immunosuppressive drugs is currently the only means to ensure long-term survival of transplanted allografts, these drugs are expensive and are associated with severe side effects (nephrotoxicity, tumor and infection development, diabetes, cardiovascular complications, etc.) that lead to substantial morbidity and mortality. Hence, any strategy capable of significantly reducing the use of immunosuppressive drugs in transplantation may have a large impact on the health and quality of life of transplant recipients.

The inventors have previously reported (see 26) that gene expression profiling employing peripheral blood specimens and oligonucleotide microarrays constitutes a high-throughput approach to dissect the biology underlying operational tolerance in human liver transplantation (3). The inventors have previously identified a set of genes whose expression varies between TOL and non-TOL. The set of genes previously identified comprised the following twenty two: transforming growth factor beta receptor III (TGFBR3, NCBI Gene ID 7049), killer cell lectin-like receptor subfamily B member 1 (KLRB1, NCBI Gene ID 3820), asparagine-linked glycosylation 8 homolog (ALG8, NCBI Gene ID 79053), Fanconi anemia complementation group G (FANCG, NCBI Gene ID 2189), gem associated protein 7 (GEMINI, NCBI Gene ID 79760), natural killer cell group 7 sequence (NKG7, NCBI Gene ID 4818), RAD23 homolog B of Saccharomyces cerevisiae (RAD23B, NCBI Gene ID 5887), SLAM family member 7 (SLAMF7, NCBI Gene ID 57823), TP53 regulated inhibitor of apoptosis 1 (TRIAP1, NCBI Gene ID 51499), protein phosphatase 1B magnesium-dependent beta isoform (PPM1B, NCBI Gene ID 5495), chromosome 10 open reading frame 119 (C10orf119, NCBI Gene ID 79892), T cell receptor delta locus (TRD@, NCBI Gene ID 6964), nucleolar protein family A member 1 (NOLA1, NCBI Gene ID 54433), DCN1 defective in cullin neddylation 1 domain containing 1 of Saccharomyces cerevisiae (DCUN1D1, NCBI Gene ID 54165), dystrobrevin binding protein 1 (DTNBP1, NCBI Gene ID 84062), N-acetylglucosamine-1-phosphate transferase alpha and beta subunits (GNPTAB, NCBI Gene ID 79158), proteasome 26S subunit non-ATPase 14 (PSMD14, NCBI Gene ID 10213), coatomer protein complex subunit zeta 1 (COPZ1, NCBI Gene ID 22818), S100 calcium binding protein A10 (S100A10, NCBI Gene ID 6281), ataxin 10 (ATXN10, NCBI Gene ID 25814), G-rich RNA sequence binding factor 1 (GRSF1, NCBI Gene ID 2926), and CD244 molecule natural killer cell receptor 2B4 (CD244, NCBI Gene ID 51744); wherein the corresponding gene expression levels above or below pre-determined cut-off levels are indicative of the tolerant state in liver transplantation. Among the previous set of genes, SLAMF7 was of particular relevance and importance. Moreover, in addition to that first set of genes, the expression of a second set can be also assessed thus improving the scope and the sensitivity of the method. That second set of genes whose expression can be additionally measured comprises the following 23:

| GENE | GENE | NCBI |
|---|---|---|
| Abbreviated | Full Name | No. |
| CTBP2 | C-terminal binding protein 2 | 1488 |
| CLIC3 | chloride intracellular channel 3 | 9022 |
| KLRF1 | killer cell lectin-like receptor subfamily F, member 1 | 51348 |
| IL2RB | interleukin 2 receptor, beta | 3560 |
| OSBPL5 | oxysterol binding protein-like 5 | 114879 |
| FEZ1 | fasciculation and elongation protein zeta 1 (zygin I) | 9638 |
| FLJ14213 | hypothetical protein FLJ14213 | 79899 |
| CD160 | CD160 molecule | 11126 |
| RGS3 | regulator of G-protein signaling 3 | 5998 |
| CX3CR1 | chemokine (C-X3-C motif) receptor 1 | 1524 |
| PTGDR | prostaglandin D2 receptor (DP) | 5729 |
| CD9 | CD9 molecule | 928 |
| PDE4B | phosphodiesterase 4B, cAMP-specific | 5142 |
| | (phosphodiesterase E4 dunce homolog, Drosophila) | |
| ERBB2 | v-erb-b2 erythroblastic leukemia viral oncogene | 2064 |
| | homolog 2, neuro/glioblastoma derived oncogene | |
| | homolog (avian) | |
| FEM1C | fem-1 homolog c (C.elegans) | 56929 |
| WDR67 | WD repeat domain 67 | 93594 |
| ZNF267 | zinc finger protein 267 | 10308 |
| ZNF295 | zinc finger protein 295 | 49854 |
| EPS8 | epidermal growth factor receptor pathway substrate 8 | 2059 |
| IL8 | interleukin 8 | 3576 |
| NCALD | neurocalcin delta | 83988 |
| NOTCH2 | Notch homolog 2 (Drosophila) | 4853 |
| RGS3 | Regulator of G-protein Signalling 3 | 5998 |
| PTCH1 | patched homolog 1 (Drosophila) | 5727 |

wherein the corresponding gene expression levels above or below predetermined cut-off levels are indicative of the tolerant state in liver transplantation. However, to use such a large number of genes presented important drawbacks.

### DESCRIPTION OF THE INVENTION

### Brief Description of the Invention

In order to solve those problems the current invention relates to the identification of genomic classifiers that would: i) comprise modest number of genes; ii) provide high diagnostic accuracy in the identification of tolerant recipients; and iii) yield reproducible results across different transcriptional platforms.

In the current invention we have employed two different gene expression profiling technologies to construct and validate a series of genomic classifiers of operational tolerance in liver transplantation. Thus, we have analyzed peripheral blood specimens from 38 adult liver transplant recipients employing oligonucleotide microarrays and quantitative real-time PCR (qPCR) and have identified several predictive models containing very low numbers of genes whose messenger RNA (mRNA) levels accurately identify operationally tolerant liver recipients. This genomic footprint of operational tolerance has been compared with gene expression patterns obtained from healthy individuals, validated on an independent cohort of 23 additional liver recipients, and employed to estimate the prevalence of tolerance among stable liver recipients receiving maintenance immunosuppressive drugs. In addition, the influence of potentially confounding clinical variables and specific PBMC subsets on tolerance related gene signatures has been thoroughly assessed. Our invention based on measurement of the expression of a modest number of genes in peripheral blood constitutes a robust non-invasive diagnostic test of operational tolerance in clinical liver transplantation.

We first analyzed peripheral blood samples obtained from operationally tolerant liver recipients and from non-tolerant recipients requiring maintenance immunosuppression employing Affymetrix microarrays. The diagnostic applicability of the resulting 26-probe genetic classifier was tested on an independent cohort of 19 stable liver transplant recipients on maintenance immunosuppression. These patients were selected according to the clinical criteria most commonly used to enrol patients in immunosuppressive weaning trials (1), and are therefore representative of the diversity of patients to whom a diagnostic test based on the identified gene signature would be applied if adopted for broad clinical use. Prediction of tolerance status based on the identified gene signature resulted in the identification of 4/19 potentially tolerant recipients (26%), which matches the prevalence of operational tolerance observed in patients selected according to the above clinical criteria (1, 5, 8). The most informative genes selected in the microarray experiments were then validated on a qPCR platform. This resulted in the identification (Table 3) of 3 qPCR-derived composite models incorporating 2, 6 and 7 genes exhibiting remarkable accuracy at discriminating TOL from Non-TOL samples in both training and independent validation sets. qPCR experiments incorporated an additional group of samples collected from healthy non-transplanted individuals (CONT). This allowed comparison of TOL and CONT expression patterns. While tolerance-related expression signatures resembled CONT more than Non-TOL, half of the genes differentially expressed between TOL and Non-TOL samples were also significantly different when comparing TOL and CONT samples. This indicates that a substantial proportion of identified genetic classifiers are very likely to be tolerance-specific. Hence, the invention relates to the selection of groups of genes, called gene signatures or fingerprints, comprising a small number of genes, allowing an accurate assessment of the tolerant state of a given subject which has been (diagnosis) or is going to be (prognosis) liver transplanted.

Accordingly, one of the embodiments of present invention deals with a method for in vitro diagnosis/prognosis of the tolerant state of a patient which, has been or is going to be, respectively, subject of a liver transplantation, comprising the steps of:
a) obtaining a biological sample from the patient and
b) measuring the expression levels in that sample of a group of genes selected among: KLRF1 (NCBI51348) and SLAMF7 (NCBI57823); or KLRF1 (NCBI51348), NKG7 (NCBI4818), IL2RB (NCBI3560), KLRB1 (NCBI3820), FANCG (NCBI2189) and GNPTAB (NCBI79158); or SLAMF7 (NCBI57823), KLRF1 (NCBI51348), CLIC3 (NCBI9022), PSMD14 (NCBI10213), ALG8 (NCBI79053), CX3CR1 (NCBI1524) and RGS3 (NCBI5998) and
c) comparing the expression fingerprint of each group of genes with the expression levels of the same group of genes of a control biological sample obtained from a non-tolerant liver transplant recipient requiring on-going immunosuppression therapy and
d) having instructions to assess tolerance or non-tolerance to liver transplantation of the patient whose biological sample has been assayed, based on the up-regulation of the expression of any of said group of genes with regard to expression threshold values for each gene of the same group of genes of the control sample.

The present invention also relates to a method for in vitro diagnosis or prognosis of the tolerant state of a patient which has been or is going to be, respectively, subject of a liver transplantation, comprising the steps of:
a) measuring the expression levels in a biological sample of said patient of a group of genes selected among: KLRF1 (NCBI51348) and SLAMF7 (NCBI57823); or KLRF1 (NCBI51348), NKG7 (NCBI4818), IL2RB (NCBI3560), KLRB1 (NCBI3820), FANCG (NCBI2189) and GNPTAB (NCBI79158); or SLAMF7 (NCBI57823), KLRF1 (NCBI51348), CLIC3 (NCBI9022), PSMD14 (NCBI10213), ALG8 (NCBI79053), CX3CR1 (NCBI1524) and RGS3 (NCBI5998) and
b) comparing the expression levels of each group of genes with the expression levels of the same group of genes in a control biological sample obtained from a non-tolerant liver transplant recipient requiring on-going immunosuppression therapy, and
c) using instructions to assess tolerance or non-tolerance to liver transplantation of the patient whose biological sample has been assayed, based on the up-regulation of the expression of any of said group of genes with regard to expression threshold values for each gene of the same group of genes of the control sample.

As used herein, “the biological sample from the patient” can be whole blood, blood cells (PBMC and particularly leukocytes), bile fluid or cells there from, urine, and can also include portions of hepatic tissue (in the form of fresh tissue, frozen sections or formalin fixed sections). As is apparent to one of ordinary skilled in the art, samples may be prepared by any available method or process depending on the subsequent analysis. Methods of isolating total mRNA are also well known. Such samples include RNA samples, but also include cDNA synthesized from a mRNA sample isolated from a cell or tissue of interest. Such samples also include DNA amplified from the cDNA, and an RNA transcribed from the amplified DNA. A preferred biological sample is the blood.

Other embodiment of present invention relates to the fact that gene expression levels are measured specifically in VδTCR+ blood cell subtype and, more particularly, that besides expression attributable to genes KLRF1 (NCBI51348) and SLAMF7 (NCBI57823), additionally, the gene expression levels of any of the following genes can also be measured: IL2RB (NCBI3560), KLRB1 (NCBI3820), CD9 (NCBI928), CD244 (NCBI51744) or CD160 (NCBI11126).

The potential impact on tolerance-related gene expression patterns of clinical variables such as age, time from transplantation, type of immunosuppressive therapy and HCV status, was specifically addressed on the microarray dataset. HCV infection had a striking impact on peripheral blood gene expression patterns, markedly outweighing the effect of tolerance itself in terms of the number of genes influenced. The effect of HCV infection on the set of genes most strongly associated with tolerance was however weak, which explains why the 26-probe microarray signature (corresponding to 23 distinct genes) could correctly identify tolerant recipients regardless of HCV infection status. Time from transplantation was found to be marginally associated with the PAM-derived 26-probe signature. This is concordant with the clinical observation that liver recipients with a longer post-transplant follow-up are more likely to become operationally tolerant (1), but clearly does not account for the expression differences between TOL and Non-TOL recipients detected in our study population. A significant effect of pharmacological immunosuppression on tolerance related gene expression patterns was excluded by the negative result of the Globaltest association analysis and by our finding that STA recipients predicted to be tolerant was grouped together with TOL recipients, which suggests that a common expression signature prevails regardless of the use of immunosuppressive drugs. Hence, we provide here a series of robust predictive models containing a strikingly small number of features capable of accurately discriminating between operationally tolerant liver recipients and those requiring ongoing pharmacological immunosuppression on the basis of peripheral blood gene expression patterns. For the widespread clinical application of a diagnostic/prognostic test based on the quantification of gene expression it is highly desirable to define a set containing a minimum number of genes. This facilitates the standardization of the test, greatly reduces its cost, and allows for the use of non high-throughput transcriptional platforms such as quantitative PCR.

In order to elucidate the PBMC subsets potentially responsible for the maintenance of the tolerant state, in the current invention we have correlated the expression levels of the most informative genes with the frequencies in peripheral blood of B cells, NK, γδTCR+, CD4+, CD8+, and CD4+CD25+ T cells. This has revealed a significant correlation of the tolerance-related gene set with both NK and γδTCR+ T cell frequencies. In addition, by employing flow cytometry immunophenotyping we have determined that among γδTCR+ T cells the Vδ1TCR+ T cell subset exhibits unique expression markers at the protein level. There are two main γδTCR+ T cell subsets in human peripheral blood: Vδ1 and Vδ2. In healthy individuals Vδ2TCR+ T cells largely predominate in peripheral blood (>80%), while Vδ1TCR+ T cells are the major subtype in tissues such as intestine, liver and spleen. In operationally tolerant liver recipients, in contrast, peripheral blood Vδ1TCR+ T cells expand and typically outnumber Vδ2TCR+ T cells (2, 3). In our present invention we have shown that Vδ1TCR+ T cells greatly influence tolerance-related transcriptional signatures. In addition, we provide evidences that peripheral blood Vδ1TCR+ T cells from tolerant liver recipients exhibit unique expression and cell surface traits that distinguish them from those present on either non-tolerant recipients or non-transplanted healthy individuals.

On the basis of gene expression and flow cytometry data presented here it is clear that tolerant liver recipients are distinct not only from recipients requiring maintenance immunosuppression, but also from non-transplanted healthy individuals.

Functional profiling of human kidney allograft tolerance employing peripheral blood samples has been previously reported by Brouard et al. (5) utilizing a two-color cDNA microarray platform (“lymphochip”) mainly containing immune-related genes (6). While it would be critical to find common features between operationally tolerant kidney and liver recipients, comparison of both studies is problematic. First, the two array platforms employed (“lymphochip” and Affymetrix U133 Plus 2.0 arrays) have only 4733 probes in common with just 543 of them being present in the SAM-derived 2482-gene list discriminating between TOL and Non-TOL liver recipients (data obtained employing the MatchMiner tool (7). This number is too low for detailed evaluation of genome-wide transcriptional similitudes, particularly when comparing two distant clinical settings and utilizing two different expression platforms. Second, the two studies analyze different patient groups (i.e. our study is focused on identifying tolerant individuals among stable liver recipients while Brouard et al. compare tolerant kidney recipients with chronic rejectors). Despite these limitations, a comparison restricted to functional pathway profiles suggests that the mechanisms accounting for operational tolerance in liver transplantation are distinct from those active in kidney recipients. Thus, operationally tolerant kidney recipients appear to be characterized by a state of immune quiescence with marked down-regulation of genes involved in lymphocyte trafficking and activation and up-regulation of genes responsible for cell cycle control (5). In contrast, in operationally tolerant liver recipients there is a manifest influence on expression patterns of cellular components of the innate immune cells while changes in pro-inflammatory pathways are barely noticeable. Furthermore, a role for B cells in liver allograft tolerance is not supported by either immunophenotyping or gene expression data, in contrast to what has been reported in kidney transplantation (8, 9).

In short, our invention reveals that measurement of the expression levels of a small set of genes in peripheral blood could be useful to accurately identify liver recipients accepting their grafts in the absence of pharmacological immunosuppression. This can be accomplished by either measuring the level of transcription of a very modest set of genes or by quantifying the expression levels of a set of surface proteins in peripheral blood Vδ1TCR+ T cells. Altogether, our invention opens the door to the possibility of withdrawing immunosuppressive drugs in recipients with high likelihood of being tolerant.

For the purpose of present invention the following non-standard abbreviations have been used: qPCR (quantitative real-time PCR); TOL (tolerant liver transplant recipient); Non-TOL (non-tolerant liver transplant recipient); STA (stable live transplant recipients under maintenance immunosuppressive therapy); SAM (significant analysis of microarrays); PAM (predictive analysis of microarrays); MiPP (misclassified penalized posterior probability algorithm); FDR (false discovery rate); EST (expressed sequence tag); HCV (hepatitis C virus).

As used in present specification the term diagnosis means the assessment of the tolerant state of a liver recipient already transplanted patient to whom an immunotherapy protocol post-surgery is required, or not. Analogously, the term prognosis means the previous assessment of the tolerant state of a patient undergoing liver transplantation before said transplantation takes place. In an advantageous embodiment, the method according to the invention is for the diagnosis of the tolerant state of a patient which has been the subject of a liver transplantation.

As used herein, the term “tolerant state” means the acceptance of a transplanted liver maintaining normal function in the absence of on-going immunosuppressive therapy. For the purposes of the current invention the terms “tolerance” and “operational tolerance” are considered as equivalent.

In an embodiment of the present invention, the gene expression levels are above pre-determined cut-off or threshold levels obtained from a control sample. In a particular embodiment, the control sample is obtained from a non-tolerant liver transplant recipient requiring on-going immunosuppression therapy that can be called immunosuppression-dependent or non-tolerant (Non-TOL). For the genes covered in the present invention the threshold values departing from which the compared gene expressions as measured in the patient's samples have to be considered up-regulated are given in Table 2. When no sign appears before the expression figure means up-expression. When sign “−” (minus) appears before the expression figures, means down-expression.

The differentially expressed genes are either up-regulated or down-regulated in a defined state. “Up-regulation” and “down-regulation” are relative terms meaning that a detectable difference (beyond the contribution of noise in the system used to measure it) is found in the amount of expression of the genes relative to some baseline. In this case, the baseline is the measured gene expression of the control sample. The genes of interest in the tolerant state are up regulated relative to the baseline level using the same measurement method.

The present invention provides means to use quantitative gene expression to diagnose tolerant liver transplant recipients before immunosuppressive drug withdrawal or reduction is attempted. The main application of this is the diagnosis of tolerant liver transplant recipients among patients receiving chronic immunosuppressive therapy. Consequently, it permits the dose reduction or discontinuation of immunosuppressive drugs in those patients identified as tolerant without undergoing rejection. This can result in a substantial decrease in the morbidity/mortality of drug-related side effects. This also means a significant decrease in the financial costs of therapy after liver transplantation.

Measuring the expression levels of the genes in the sample can be carried out over the transcripts of these genes (messenger RNA) or over the translation products, i.e. the proteins. Means for measuring the gene expression must be taken in its broader sense, as any available commercial mean comprising any nucleic acid capable of hybridization which, in turn, might be detected by any available mean, with the gene DNA or mRNA transcripted therefrom. Means for measuring gene expression, for the purpose of present invention, cover also any available and commercial mean suitable for detecting the proteins encoded by the genes whose expression is the base of the method and kit of invention.

In a particular embodiment, measuring the gene expression levels is carried out using a microarray or a gene chip which comprises nucleic acid probes. Said nucleic acid probes comprise sequences that specifically hybridize to the transcripts of the set of genes defined above. At least one probe for each of the transcript must be on the microarray or the gene chip for detecting all the genes defined above, but it is possible to have more than one probe for the same transcript.

The term “specifically hybridize to” refers to the binding, duplexing, or hybridizing of a molecule substantially to or only to a particular nucleotide sequence or sequences under stringent conditions when that sequence is present in a complex mixture (e.g., total cellular DNA or RNA). “Hybridization” refers to the process in which two single-stranded polynucleotides bind non-covalently to form a stable double-stranded polynucleotide.

In any method according to the invention, the step of measuring expression levels of the indicated sets of genes is always performed in vitro.

Microarray technology measures mRNA levels of many genes simultaneously thereby presenting a powerful tool for identifying gene expression profiles for a disease or a specific state. Two microarray technologies are currently in wide use. The first are complementary DNA (cDNA) microarrays and the second are oligonucleotide microarrays. Although differences exist in the construction of these chips, essentially all downstream data analysis and output are the same. Typically, a nucleic acid sample is prepared from appropriate source and labeled with a signal moiety, such as a fluorescent label. The sample is hybridized with the microarray under appropriate conditions. The microarrays are then washed or otherwise processed to remove non-hybridized sample nucleic acids. The hybridization is then evaluated by detecting the distribution of the label on the chip. The distribution of label may be detected by scanning the microarrays to determine fluorescence intensity distribution. Typically, the hybridization of each probe is reflected by corresponding pixel intensities. The signal intensity is proportional to the cDNA amount, and thus mRNA, expressed in the sample. Analysis of the differential expression levels is conducted by comparing such intensities for the test sample and for the control sample. A ratio of these intensities indicates the fold-change in gene expression between the test and control samples.

In a particular embodiment of the invention, the microarray is a cDNA microarray. In this format, probes of cDNA (˜500-5000 bases long) are immobilized to a solid surface, e.g., glass, using robot spotting and exposed to a set of targets either separately or in a mixture. This method, traditionally called DNA microarray, was developed at Stanford University.

In another particular embodiment, the microarray is an oligonucleotide microarray. In this format, oligonucleotides (˜20-80-mer) or peptide nucleic acid (PNA) probes are synthesized either in situ (on-chip) or by conventional synthesis followed by on-chip immobilization. The microarray is exposed to labeled sample DNA, hybridized, and the identity/abundance of complementary sequences is determined. This method, historically called DNA chip, was developed by Affymetrix, Inc., which sells its photolithographically fabricated products under the GeneChip® trademark. Many companies are manufacturing oligonucleotide based chips using alternative in-situ synthesis or depositioning technologies.

The microarray can assume a variety of formats, e.g., libraries of soluble molecules; and libraries of compounds tethered to resin beads, silica chips, on glass or other solid supports. A number of different microarray configurations, supports and production methods are known to those skilled in the art. Probes may be prepared by any method known in the art, including synthetically or grown in a biological host. Synthetic methods include but are not limited to oligonucleotide synthesis, riboprobes, and polymerase chain reaction (PCR). The probes may be labeled with a detectable marker by any method known in the art. Methods for labeling probes include random priming, end labeling and PCR and nick translation.

Preferably, microarrays used for performing the methods according to the invention are specifically designed for this purpose, so that they comprise mainly probes that specifically hybridize with the selected set of genes. In particular, while such microarrays may comprise probes specific for other genes (in particular control probes, see below), they preferably comprise probes specific for no more than 500 distinct genes, more preferably no more than 400, 300, 250, 200, 150, 100, even more preferably no more than 90, 80, 70, 60, 50, 45, 40, 35, 30, 25, 20, 10 or even 7 distinct genes or even 6 distinct genes, or even 2 distinct genes among which are the selected set of genes.

In a particular embodiment, the microarray or the gene chip further comprises one or more internal control probes that act for example, as normalization control probes, expression level control probes and mismatch control probes. Normalization controls provide a control for variations in hybridization conditions, label intensity, “reading” efficiency and other factors that may cause the signal of a perfect hybridization to vary between microarrays.

Expression level controls are probes that hybridize specifically with constitutively expressed genes in the analyzed sample (“housekeeping genes”). Mismatch controls are oligonucleotide probes identical to their corresponding test or control probes except for the presence of one or more mismatched bases. Mismatch probes thus provide a control for non-specific binding or cross hybridization to a nucleic acid in the sample other than the target to which the probe is directed (false positives).

In other embodiments of the invention, measuring the gene expression levels of the genes is carried out by reverse transcription PCR (RT-PCR), competitive RT-PCR, real time RT-PCR, differential display RT-PCR, Northern Blot analysis and other related tests. In a particular embodiment of the invention, measuring the gene expression levels is carried out by quantitative reverse transcription PCR of RNA extracted from the sample. In a more particular embodiment, the RT-PCR comprises one or more internal control reagents. Another option is to conduct these techniques of gene expression quantification using PCR reactions, to amplify cDNA or cRNA produced from mRNA and analyze it via microarray. In another embodiment, measuring the gene expression levels of the genes is carried out using isothermal amplification. According to the present invention, the expression “isotherm amplification” covers any DNA amplification technology which does not resort to thermal cycling. Examples of such isotherm amplification technologies include NASBA (nucleic acid sequence-based amplification, see 27), 3SR (self-sustained sequence replication, see 28), and LAMP (loop-mediated isothermal amplification, see 29).

3SR and NASBA eliminate heat denaturation by using a set of transcription and reverse transcription reactions to amplify the target sequence. NASBA is a primer-dependent technology that can be used for the continuous amplification of nucleic acids in a single mixture at one temperature. In 3SR, a target nucleic acid sequence can be replicated (amplified) exponentially in vitro under isothermal conditions by using three enzymatic activities essential to retroviral replication: reverse transcriptase, RNase H, and a DNA-dependent RNA polymerase. By mimicking the retroviral strategy of RNA replication by means of cDNA intermediates, this reaction accumulates cDNA and RNA copies of the original target.

LAMP technology employs a DNA polymerase and a set of four specially designed primers that recognize a total of six distinct sequences on the target DNA. An inner primer containing sequences of the sense and antisense strands of the target DNA initiates LAMP. The following strand displacement DNA synthesis primed by an outer primer releases a single-stranded DNA. This serves as template for DNA synthesis primed by the second inner and outer primers that hybridize to the other end of the target, which produces a stem-loop DNA structure. In subsequent LAMP cycling one inner primer hybridizes to the loop on the product and initiates displacement DNA synthesis, yielding the original stem-loop DNA and a new stem-loop DNA with a stem twice as long. The cycling reaction continues with accumulation of 10⁹ copies of target in less than an hour. The final products are stem-loop DNAs with several inverted repeats of the target and cauliflower-like structures with multiple loops formed by annealing between alternately inverted repeats of the target in the same strand (29).

In other embodiments, measuring the gene expression levels is carried out by detecting protein encoded by each of the genes with antibodies specific to the proteins or by a proteins chip. A protein chip or a protein microarray can assume a variety of formats, but commonly consists of a solid surface onto which enzymes, receptor proteins, antibodies or small molecules are immobilized and used as probes to detect proteins contained in the target sample. In another embodiment, measuring the gene expression levels is carried out by HPLC. Gene expression can also be detected by measuring a characteristic of the gene that affects transcriptional activity of the gene, such as DNA amplification, methylation, mutation and allelic variation. Such methods are known to those skilled in the art.

Other aspects of the invention are kits for conducting the assays described above. Since kits are based on the selection of a set of genes comprising the ones described above, kits are simpler and cheaper than others based on a large amount of genes, such as many commercial microarrays with thousands of probes. Thus, an aspect of the invention refers to the use of a kit for performing the method as defined above, comprising (i) means for measuring the gene expression levels of the selected genes; and (ii) instructions for correlating the gene expression levels above or below pre-determined cut-off levels indicative of the tolerant state in liver transplantation. In such a kit, the means for measuring the gene expression levels of the selected genes may in some cases permit to measure the expression of additional genes. However, said means preferably permit to measure the expression of no more than 500 distinct genes, more preferably no more than 400, 300, 250, 200, 150, 100, even more preferably no more than 90, 80, 70, 60, 50, 45, 40, 35, 30, 25, 20, 10 or even 7 distinct genes, or even 6 distinct genes or even 2 distinct genes among which are the selected set of genes.

In a particular embodiment of the invention, the means comprise a microarray or a gene chip which comprises nucleic acid probes, said nucleic acid probes comprising sequences that specifically hybridize to the transcripts of the set of genes defined above. As mentioned before, such a microarray preferably comprises nucleic acid probes specific for no more than 500 distinct genes, more preferably no more than 400, 300, 250, 200, 150, 100, even more preferably no more than 90, 80, 70, 60, 50, 45, 40, 35, 30, 25, 20, 10 or even 7 distinct genes, or even 6 distinct genes or even 2 distinct genes among which are the selected set of genes. Additionally, the kit further comprises reagents for performing the microarray analysis.

In another embodiment, the means comprise oligonucleotide primers for performing a quantitative reverse transcription PCR, said primers comprising sequences that specifically hybridize to the complementary DNA derived from the transcripts of the set of genes defined above. Here also, the kit preferably comprises primers with sequences that specifically hybridize to no more than 500 distinct genes, more preferably no more than 400, 300, 250, 200, 150, 100, even more preferably no more than 90, 80, 70, 60, 50, 45, 40, 35, 30, 25, 20, 10 or even 7 distinct genes, or even 6 distinct genes or even 2 distinct genes, among which are the selected set of genes.

Each such kit would preferably include instructions as well as the reagents typical for the type of assay described. These can include, for example, nucleic acid arrays (e.g. cDNA or oligonucleotide microarrays), as described above, configured to discern the gene expression profile of the invention. They can also contain reagents used to conduct nucleic acid amplification and detection including, for example, reverse transcriptase, reverse transcriptase primer, a corresponding PCR primer set, a thermostable DNA polymerase, such as Taq polymerase, and a suitable detection reagent(s), such as, among others, fluorescent probes or dyes that bind to double-strand DNA such as ethidium bromide or SYBRgreen. Antibody based kits will contain buffers, secondary antibodies, detection enzymes and substrate, e.g. Horse Radish Peroxidase or biotin-avidin based reagents.

The invention relates not only to the use of such kits, but also to kits as described above themselves.

Another aspect of the invention refers to the use of a microarray or a gene chip for performing the method as defined above, comprising a solid support and displayed thereon nucleic acid probes which comprises sequences that specifically hybridize to the transcripts of the set of genes defined above. Here also, said microarray preferably comprises nucleic acid probes specific for no more than 500 distinct genes, more preferably no more than 400, 300, 250, 200, 150, 100, even more preferably no more than 90, 80, 70, 60, 50, 45, 40, 35, 30, 25, 20, 10 or even 7 distinct genes, or even 6 distinct genes or even 2 distinct genes among which are the selected set of genes.

The practice of the present invention may also employ conventional biology methods and software. Computer software products of the invention typically include computer readable medium having computer-executable instructions for performing the logic steps of the method of the invention. The present invention may also make use of various computer program products and software for a variety of purposes, such as probe design, management of data, analysis, and instrument operation.

One important aspect of the invention is to carry out the method of the invention in the particular blood cell subtype Vδ1TCR+, which is mostly present among the tolerant group of patients in opposition to cell subtype Vδ2TCR+, which is the one more common in the non-tolerant group.

Finally, another aspect of the invention refers to a method for selecting or modifying a treatment protocol, either before or after liver transplantation is performed, comprising the use of the method of assessing diagnosis and/or prognosis as defined above. Before liver transplantation, the invention permits to identify those patients that will eventually develop tolerance and therefore, can benefit from less aggressive immunosuppression strategies. If liver transplantation has already been done, the invention permits to adequate therapy to the patient status. Patient's therapy can be altered as with additional therapeutics, with changes to the dosage or to the frequency, or with elimination of the current treatment. Such analysis permits intervention and therapy adjustment prior to detectable clinical indicia or in the face of otherwise ambiguous clinical indicia. Thus, preferably, the method according to the invention is for modifying a treatment protocol, after liver transplantation has been performed.

In a particular embodiment, the invention concerns a method for selecting a treatment protocol for a patient before liver transplantation is performed, comprising:
a) prognosing the tolerant or non-tolerant state of said patient using the method according to the invention as described above, and
b) if said patient is prognosed as tolerant, then selecting a less aggressive immunosuppressive treatment compared to standard immunosuppressive protocols,
otherwise, selecting a standard immunosuppressive protocol.

In another embodiment, the invention concerns a method for modifying a treatment protocol for a patient after liver transplantation has been performed, comprising:
c) diagnosing the tolerant or non-tolerant state of said patient using the method according to the invention as described above, and
d) if said patient is diagnosed as tolerant, then decreasing the treatment dosage or frequency, replacing the treatment by a less aggressive drug, or even stopping the treatment,
otherwise, maintaining the treatment or adding a new immunosuppressive treatment.

While the particular genes sets described in the application for use in diagnosis or prognosis of a tolerant state in a liver-transplanted patient are particularly sensitive and specific, acceptable set of genes might be obtained by a minor modification. In particular, the addition of a few additional genes (1 to 3) or the replacement of one or more genes by other genes may result in acceptable sets of genes.

Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. The following examples are provided by way of illustration, and they are not intended to be limiting of the present invention.

### DESCRIPTION OF THE FIGURES

FIG. 1: Process outline. Peripheral blood samples were obtained from a total of 80 liver transplant recipients and 16 healthy individuals. Samples from operationally tolerant (TOL) and non-tolerant (Non-TOL) recipients were separated into a training set (38 samples) and a test set (23 samples). Differential microarray gene expression between TOL and Non-TOL samples in the training set was first estimated employing SAM. This was followed by a search to identify genetic classifiers for prediction employing PAM, which resulted in a 26-probe signature (corresponding to 23 distinct genes). The PAM-derived signature was then employed to estimate the prevalence of tolerance among a cohort of 19 STA recipients. Next, among the genes identified by SAM and PAM 68 genes were selected for validation on a qPCR platform to which were added some genes from the literature, and the 34 validated targets were employed to identify additional classifiers employing MiPP. The 3 signatures identified by MiPP on the qPCR data set were then used to classify samples in the independent test of 11 TOL and 12 Non-TOL recipients. None of the samples from the test set were employed for the genetic classifier discovery process.

FIG. 2: Differential gene expression between TOL and Non-TOL samples. A)

Expression profiles of the 100 most significant genes among the 2482 probes identified by SAM. Results are expressed as a matrix view of gene expression data (“heatmap”) where rows represent genes and columns represent hybridized samples. The intensity of each colour denotes the standardized ratio between each value and the average expression of each gene across all samples. Red coloured pixels correspond to an increased abundance of the mRNA in the indicated blood sample, whereas green pixels indicate decreased mRNA levels. B) Bar plot showing the results obtained by Globaltest for individual probes selected by PAM. Bar height above the reference line corresponds to a statistically significant association with tolerance. Red colour represents negative association, green colour positive association. C) Multidimensional scaling of TOL (▴) and Non-TOL () samples according to the expression of the 26 probes selected by PAM. Distances between samples plotted in the three-dimensional graph are proportional to their dissimilarities in gene expression. TOL and Non-TOL samples appear as two well-defined and clearly separated groups.

FIG. 3: Estimation of potentially tolerant individuals among stable liver recipients under maintenance immunosuppressive drugs. A) STA recipients classified as tolerant (STA-Affy TOL) exhibit higher levels of Vδ1 TCR+ T cells and Vδ1/Vδ2 T cell ratio than either STA recipients classified as non-tolerant (STA-Affy Non-TOL) or CONT individuals. B) Multidimensional scaling plot incorporating TOL (▴) and Non-TOL () samples together with STA samples classified as either tolerant (STA-Affy TOL; Δ) or non-tolerant (STA-Affy Non-TOL; ∘) on the basis of the expression of the 26 microarray probes corresponding to the 23 microarray genes selected by PAM. Distances between samples plotted in the three-dimensional graph are proportional to their dissimilarities in gene expression.

FIG. 4: qPCR validation of selected microarray gene expression measurements. A) Heatmap representing the expression profiles of genes with significant differential expression when comparing TOL versus Non-TOL, and TOL versus CONT samples (ttest P<0.05). The intensity of each colour denotes the standardized ratio between each value and the average expression of each gene across all samples. Red coloured pixels correspond to an increased abundance of the mRNA in the indicated blood sample, whereas green pixels indicate decreased mRNA levels. The checkerboard plot on the left represents the statistical significance of TOL vs Non-TOL and TOL vs CONT comparisons, with black squares corresponding to P-value <0.05 by t-test. B) Multidimensional scaling plot incorporating TOL (▴), Non-TOL () and CONT ( ) samples. Distances between samples plotted in the three-dimensional graph are proportional to their dissimilarities in gene expression as assessed by qPCR. CONT samples cluster between TOL and Non-TOL samples.

FIG. 5: Impact of hepatitis C virus (HCV) infection and PBMC subsets on global gene expression measurements. A) Venn diagram representing the number of statistically significant genes between TOL and Non-TOL samples stratified on the basis of HCV infection status (SAM; FDR<0.05). B) Bar plot showing the influence of HCV infection (upper panel) and tolerance (lower panel) on the individual 26 probes, corresponding to the 23 genes, selected by PAM according to Globaltest. Bar height above the reference line corresponds to a statistically significant association. Red colour represents negative association, green colour positive association. C) Checkerboard plot representing the correlation between PBMC subset frequency and the expression of the individual 26 probes, corresponding to the 23 genes, selected by PAM. Results are shown as a matrix where white squares correspond to non-significant associations and black squares significant associations (P-value <0.05) according to Globaltest. For comparison, tolerance and HCV status have been included in the analysis as well.

FIG. 6: Differences in protein expression in peripheral mononuclear between TOL, Non-TOL and CONT recipients. A) Expression of ILRB2, KLRB1, CD244, CD9, KLRF1, CD160 and SLAMF7 on peripheral blood mononuclear cells. Representative flow cytometry histograms showing protein expression on TOL, Non-TOL and CONT samples. B) Differences in protein expression levels between TOL, Non-TOL and CONT samples. Bar plots represent mean expression (% of positive cells or mean fluorescence intensity (MFI) depending on the marker analysed) +/−SD from 6 TOL, 6 Non-TOL and 5 CONT samples. (*)=P-value <0.05 (t-test) between TOL and Non-TOL; (**)=P-value <0.05 (t-test) between TOL and CONT.

### DETAILED DESCRIPTION OF THE INVENTION

The present invention is illustrated by the following examples:

### Example 1

### Patients

Peripheral blood samples were collected from a cohort of 28 operationally tolerant liver transplant recipients (TOL) and 33 liver recipients in whom drug weaning was attempted but led to acute rejection requiring reintroduction of immunosuppressive drugs (non-tolerant, Non-TOL). TOL recipients had been intentionally weaned from immunosuppressive therapy under medical supervision. Criteria employed to select patients for immunosuppression weaning in the participating institutions were the following: a) >3 years after transplantation; single drug immunosuppression; b) absence of acute rejection episodes in the previous 12 months; absence of signs of acute/chronic rejection in liver histology; and c) absence of autoimmune liver disease before or after transplantation. In TOL recipients blood was collected >1 year after successful immunosuppressive drug discontinuation, while in Non-TOL recipients specimens were harvested >1 year after complete resolution of the acute rejection episode (at the time of blood collection all Non-TOL recipients had normalized liver function tests and were receiving low dose immunosuppression in monotherapy). Additionally, peripheral blood samples were also obtained from 16 age-matched healthy controls (CONT), and 19 stable liver transplant recipients on maintenance immunosuppression (STA) that fulfilled the aforementioned clinical criteria for drug weaning In patients fulfilling these criteria the prevalence of operational tolerance ranges between 20 and 30% (5, 8). Clinical and demographic characteristics of patients included in the study are summarized in Table 1. The study was accepted by the Institutional Review Boards of all participating institutions, and informed consent was obtained from all patients. A report containing blood cell immunophenotyping findings together with preliminary microarray gene expression data obtained from a subset of the patients enrolled in the current study has been recently published (3).

### Example 2

### Microarray Experiments

Microarray experiments were conducted on PBMCs obtained from 21 Non-TOL, 17 TOL and 19 STA recipients. PBMCs were isolated employing a Ficoll-Hypaque layer (Amersham Biosciences), total RNA was extracted with Tryzol reagent (Life Technologies), and the derived cDNA samples were hybridized onto Affymetrix Human Genome U133 Plus 2.0 arrays containing 54675 probes for 47000 transcripts (Affymetrix). Sample handling and RNA extraction was performed by the same investigator in all cases (M.M-L1).

### Example 3

### Microarray Data Normalisation

Microarray data from 57 samples (21 Non-TOL, 17 TOL and 19 STA) were normalised using the GC content adjusted-robust multi-array (GC-RMA) algorithm, which computes expression values from probe intensity values incorporating probe sequence information (10). Next we employed a conservative probe-filtering step excluding those probes not reaching a log 2 expression value of 5 in at least 1 sample, which resulted in the selection of a total of 23782 probes out of the original 54675 set. In order to eliminate non-biological experimental variation or batch effects observed across successive batches of microarray experiments we applied ComBat approach, which uses nonparametric empirical Bayes frameworks for data adjustment (11).

### Example 4

### Differential Expression Assessment and Prediction

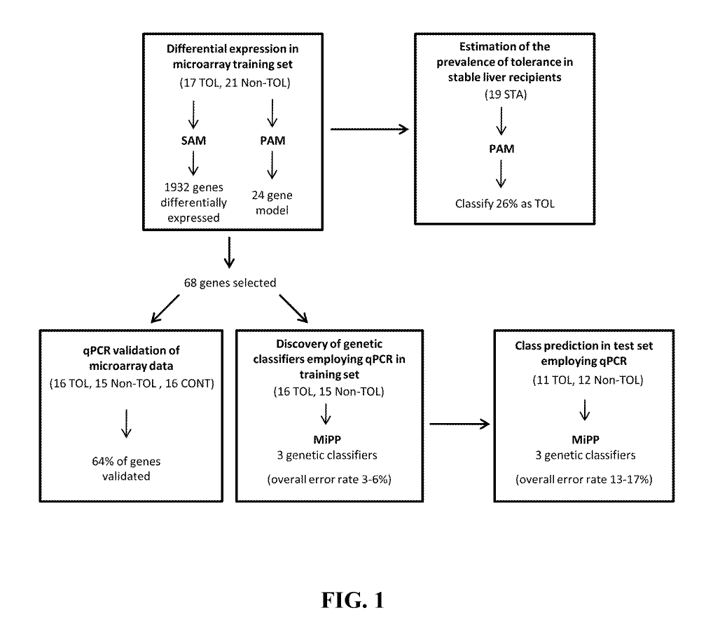

An outline of the study design is depicted in FIG. 1. We first used Significant

Analysis of Microarray (SAM) (12) to identify genes differentially expressed between the TOL and Non-TOL groups (17 and 21 samples respectively) within the filtered 23782-probe set. SAM uses modified t test statistics for each gene of a dataset and a fudge factor to compute the t value, thereby controlling for unrealistically low standard deviations for each gene. Furthermore SAM allows control of the false discovery rate (FDR) by selecting a threshold for the difference between the actual test result and the result obtained from repeated permutations of the tested groups. For the current study we employed SAM selection using FDR <5% and 1000 permutations on 3 comparison groups: TOL versus Non-TOL, TOL hepatitis C virus infection positive (HCV-pos) versus Non-TOL HCV-pos, and TOL hepatitis C virus infection negative (HCV-neg) versus Non-TOL HCV-neg. Differential gene expression was further explored by using the nearest shrunken centroid classifier implemented in the Predictive Analysis of Microarray (PAM) (13) package to identify within the 23782-probe set the minimal set of genes capable of predicting the tolerant state with an overall error rate (ER) <5%. This method incorporates an internal cross-validation step during feature selection in which the model is fit on 90% of the samples and then the class of the remaining 10% is predicted. This procedure is repeated 10 times to compute the overall error (ten-fold cross-validation). The PAM classifier was then used on the 38-sample set to perform multidimensional scaling analysis on the basis of between-sample Euclidean distances as implemented by the isoMDS function in R. This method is capable of visualizing high dimensional data (such as multiple expression measurements) in a three dimensional graph in which the distances between samples are kept as unchanged as possible. Finally, the PAM classifier was employed to predict class in the set of 19 samples obtained from STA patients.

### Example 5

### Correlation of Microarray Data with Clinical Variables and PBMC Subsets

The Globaltest algorithm (14) from the Bioconductor package (www.bioconductor.org) was employed to test if potentially confounding clinical variables such as patient age, gender, time from transplantation, hepatitis C virus (HCV) status, immunosuppressive therapy (tacrolimus, cyclosporine A or mycopheno late mophetil) and peripheral blood monocyte, lymphocyte, and neutrophil counts could be influencing gene expression levels. The same strategy was employed to estimate the correlation between microarray expression data and the proportion of peripheral blood CD4+CD25+, CD4+Foxp3+, CD4+, CD8+, CD19+, NKT, total γδTCR+, Vδ1TCR+ and Vδ2TCR+ T cells. Globaltest is a method to determine if the expression pattern of a pre-specified group of genes is related to a clinical variable, which can be either a discrete variable or a continuous measurement. This test is based on an empirical Bayesian generalized linear model, where the regression coefficients between gene expression data and clinical measurements are random variables. A goodness of fit test is applied on the basis of this model. The Globaltest method computes a statistic Q and a P-value to measure the influence of our group of genes on the clinical variable measured. For each probe, the influence (Q) in predicting measured clinical variable is estimated against the expected value, and ranked among the probes under study. The weight of each probe is also assessed by the z-score considering the standard deviation of each probe in all samples used in the analysis.

### Example 6

### Quantitative Real-Time PCR Experiments

The expression pattern of a group of target genes and 4 housekeeping genes (18S, GUS, HPRT1 and GAPDH) was measured by quantitative real-time PCR (qPCR) employing the ABI 7900 Sequence Detector System and LDA microfluidic PCR cards (PE Applied Biosystems, Foster City, Calif., USA) on peripheral blood samples obtained from 15 Non-TOL, 16 TOL and 16 CONT individuals. Selected target genes included the 23 genes identified by PAM, 44 genes selected among those most highly ranked in the SAM-derived gene list, and 6 genes (UBD, HLA-DOB, FOXP3, LTBP3, MAN1A1, LGALS3) selected on the basis of previous reports (3, 5, 8). To quantify the levels of mRNA we normalized the expression of the target genes to the housekeeping gene HPRT1 (which was found to be the most stably expressed gene among the 4 housekeeping genes selected) and presented the results as relative expression between cDNA of the target samples and a calibrated sample according to the ΔΔCT method. All qPCR experiments were performed in duplicates. Total RNA was treated with DNAse reagent (Ambion), and reverse transcription performed using Multiscribed Reverse Transcriptase Enzyme (PE Applied Biosystems). Results were analyzed employing standard two-class unpaired t-test. Reproducibility of gene expression measurements was assessed by comparing inter-patient and inter-assay variation in a set of qPCR experiments that included 22 genes and samples from 16 recipients. For this experiment two peripheral blood samples collected at 2 separated time-points (mean 57 days, range 11-244 days) were employed. Inter-assay variation was defined as the variation between PCR runs carried out employing the two different peripheral blood samples from the same patient. To construct classification models containing a minimal set of features (genes) with the lowest possible classification error both in training and independent test sets we employed the misclassification penalized posterior (MiPP) algorithm (17) on the 34 target genes differentially expressed between TOL and Non-TOL samples (ttest P<0.05). MiPP is a recently developed method for assessing the performance of a prediction model that computes the sum of the posterior classification probabilities penalized by the number of incorrectly classified samples. The MIPP application performs an exhaustive search for gene models by sequentially selecting the most predictive genes and automatically removing the selected genes in subsequent runs. For our analysis we conducted 10 sequential runs and employed all predictive algorithms included in the MIPP application (linear discriminant analysis, quadratic discriminant analysis, support vector machine learning, and logistic regression). Internal computational validation was performed employing both 10-fold cross-validation and random-split validation (number of splits=100). The composite models obtained were then employed to predict tolerance in the independent test set of 11 TOL and 12 Non-TOL samples from which no microarray data were available. The three models with a lower classification error rate (in training set and test set) were selected.

### Example 7

### Peripheral Blood Immunophenotyping

Flow cytometry immunophenotyping data from PBMCs obtained from 16 TOL and 16 Non-TOL recipients have been reported elsewhere (3). In the current study we assessed the proportion of CD4+CD25+, CD4+Foxp3+, total γδTCR+, δ1 γδTCR+, δ2 γδTCR+, CD19+, NK and NKT cell subsets on peripheral blood specimens obtained from 19 STA recipients and from 1 TOL and 5 Non-TOL recipients (from whom no previous data were available). Immunophenotyping results from all 57 recipients were employed to correlate PBMC subset frequencies with microarray expression data. Foxp3 fluorescent monoclonal antibodies were purchased from from eBioscience. All remaining antibodies were purchased from BD Biosciences.

### Example 8

### Candidate Gene Discovery and Internal Validation of Microarray Data

To assess differential gene expression between tolerant and non-tolerant recipients oligonucleotide microarray experiments were conducted on PBMCs obtained from 17 TOL and 21 Non-TOL recipients (Table 1 and FIG. 1).

TABLE 1
Demographic characteristics of patient groups.

| | | | Time from | Time from | | | |
|---|---|---|---|---|---|---|---|
| | | | transplantation | weaning | HCV | | |
| Clinical diagnosis | Number | Age ^{A} | (years) ^{A} | (years) ^{A} | infection ^{A} | Treatment ^{B} | Center ^{C} |
| Operationally tolerant (TOL) | 28 | 57 (40-68) | 10.9 (4-16) | 5.6 (1-8) | 21% | | |
| Non-Tolerant (Non-TOL) | 33 | 53 (39-67) | 8.2 (4-15) | | 25% | | |
| Training set | | | | | | | |
| TOL | 17 | 55 | 10.39 | 7.52 | 18% | | B, R, M, L |
| Non-TOL | 21 | 52 | 9.45 | | 29% | 48% CsA, 38% FK, | B, R, M, L |
| | | | | | | 9% MMF, 5% SRL | |
| Test set | | | | | | | |
| TOL | 11 | 61 | 11.7 | 2.6 | 27% | | B, R, L |
| Non-TOL | 12 | 55 | 6 | | 17% | 25% MMF, 50% FK, | B, R, L |
| | | | | | | 25% CsA | |
| Stable recipients (STA) | 19 | 55 (45-74) | 9 (5-12) | | 13% | 40% CsA, 30% FK, | B |
| | | | | | | 30% MMF | |
| Healthy controls (CONT) | 16 | 62 (42-70) | | | | | B |

^{A} Mean (range)
^{B} CsA, cyclosporine A; FK, tacrolimus; MMF, mycophenolate mophetil; SRL, sirolimus (all patients were receiving immunosuppressive drugs in monotherapy).
^{C} B, Hospital Clinic Barcelona, Spain; R, University Tor Vergata Rome, Italy; M, Hospital Virgen de Arrixaca Murcia, Spain; L, Catholic University Louvain, Belgium.

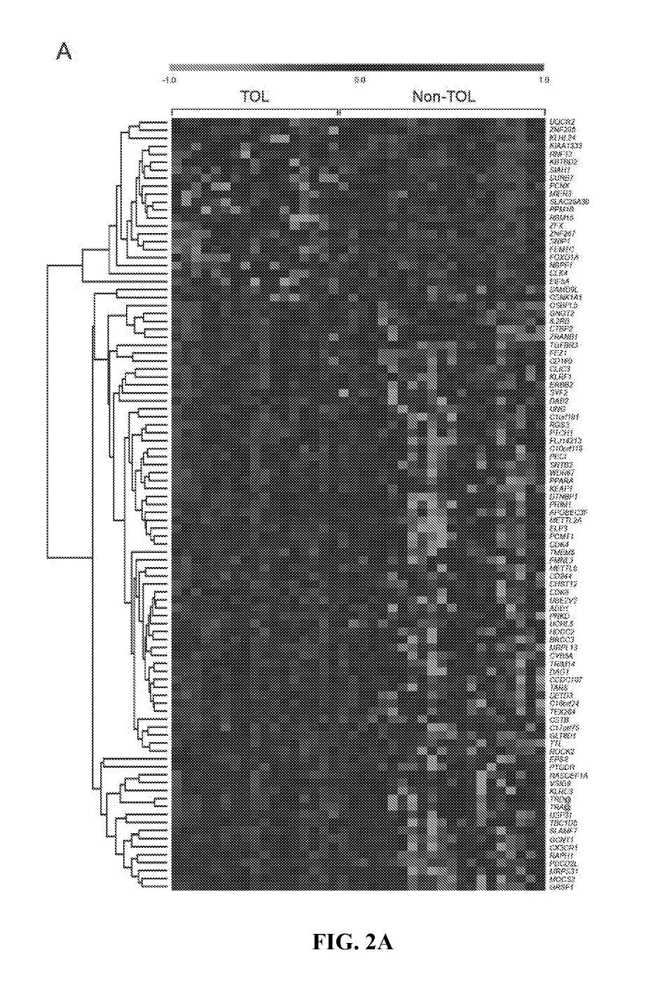

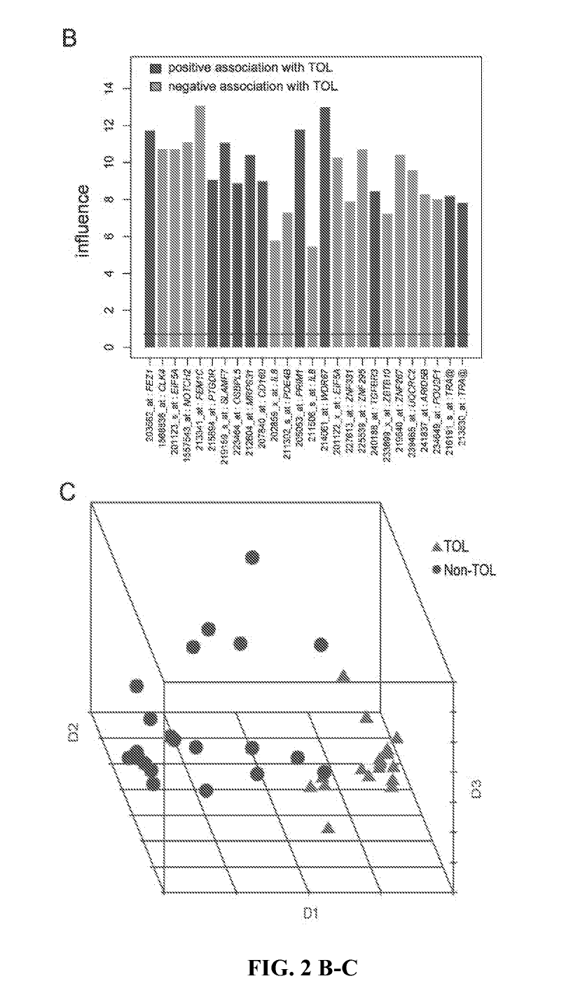

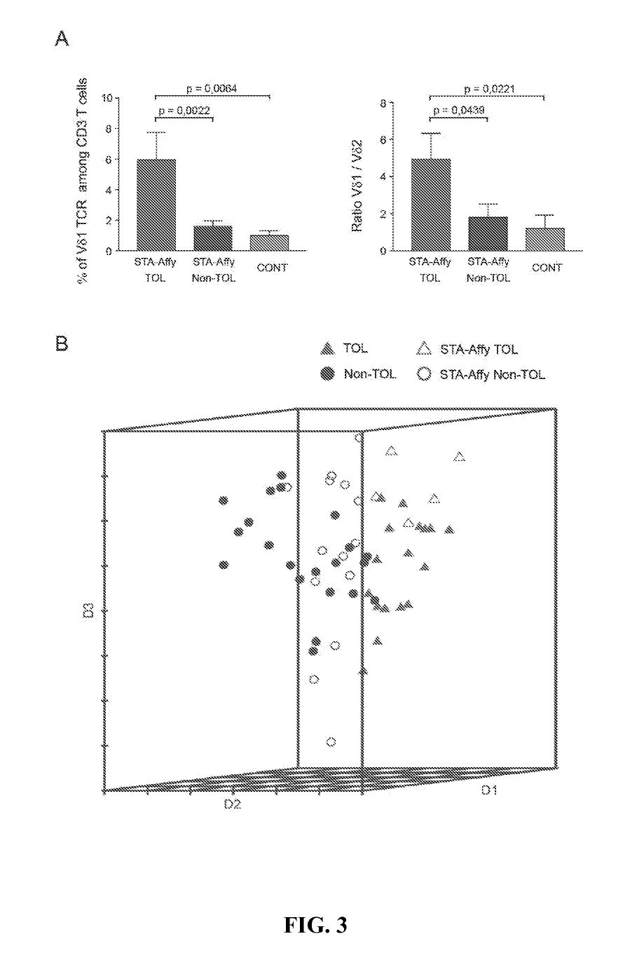

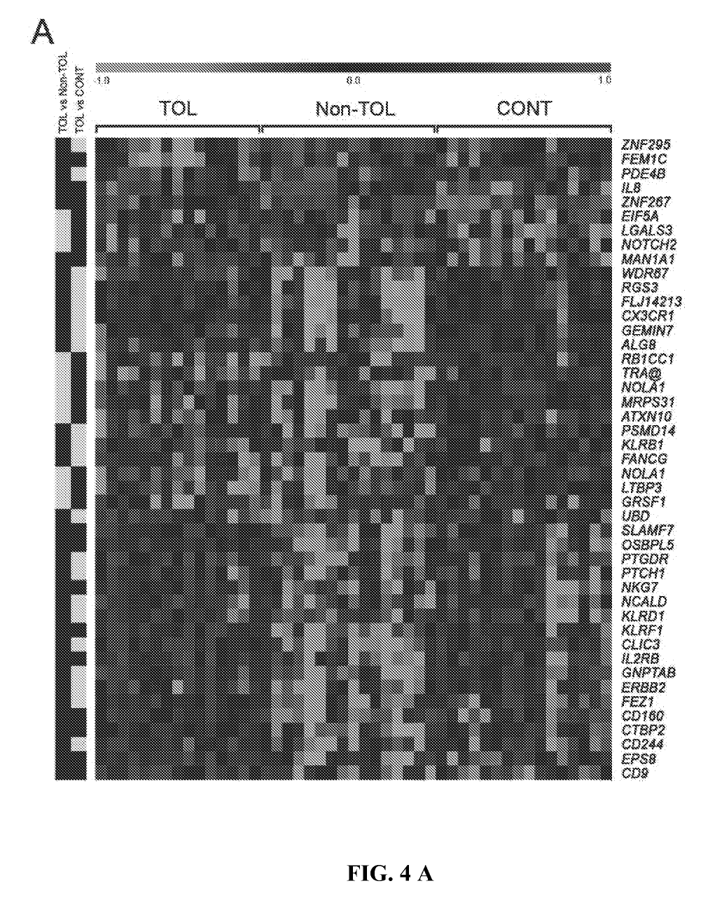

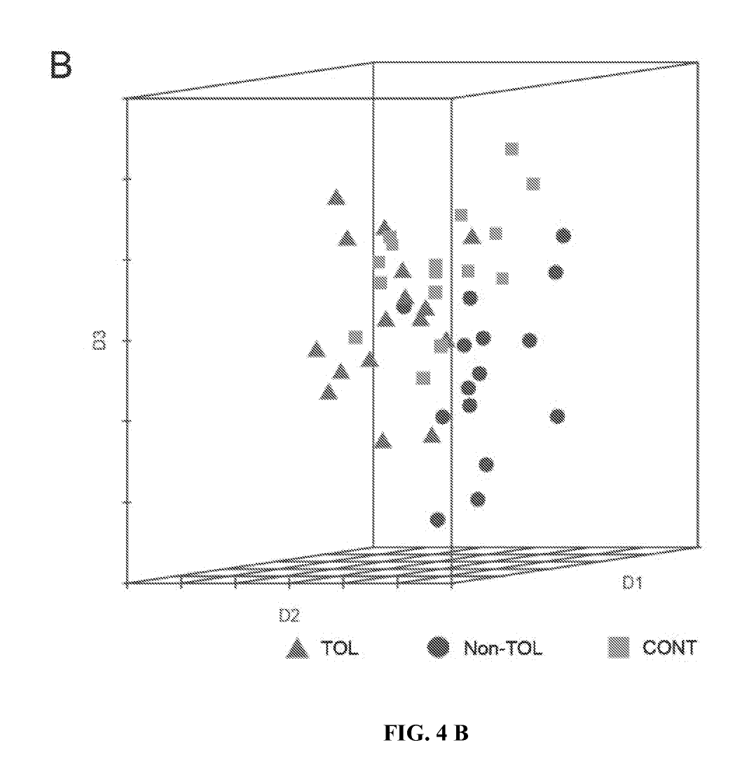

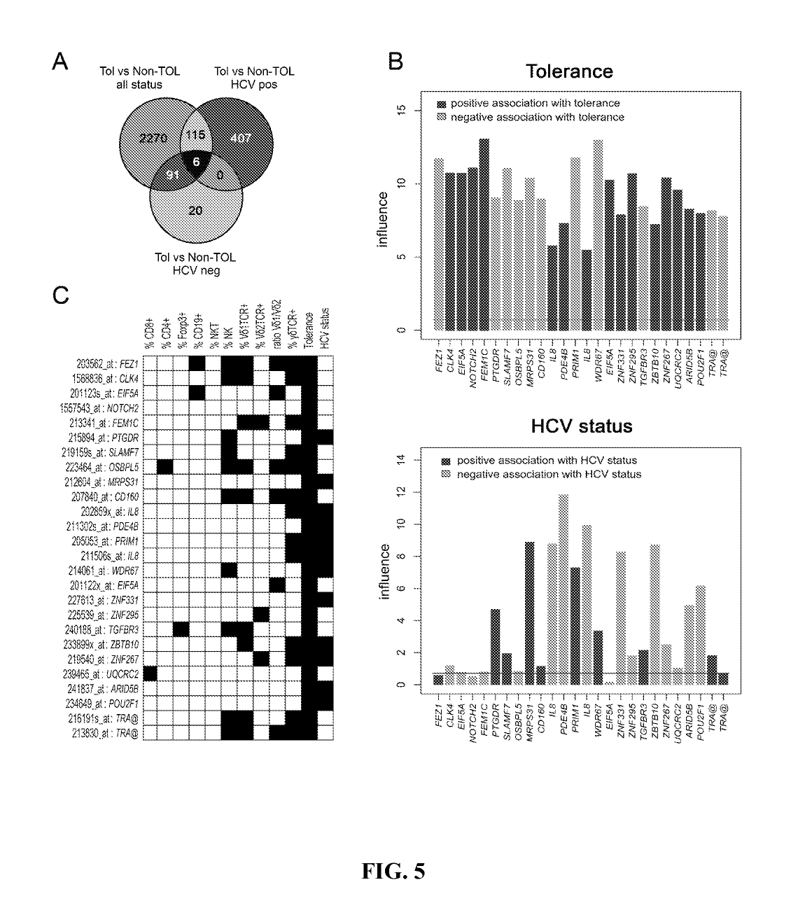

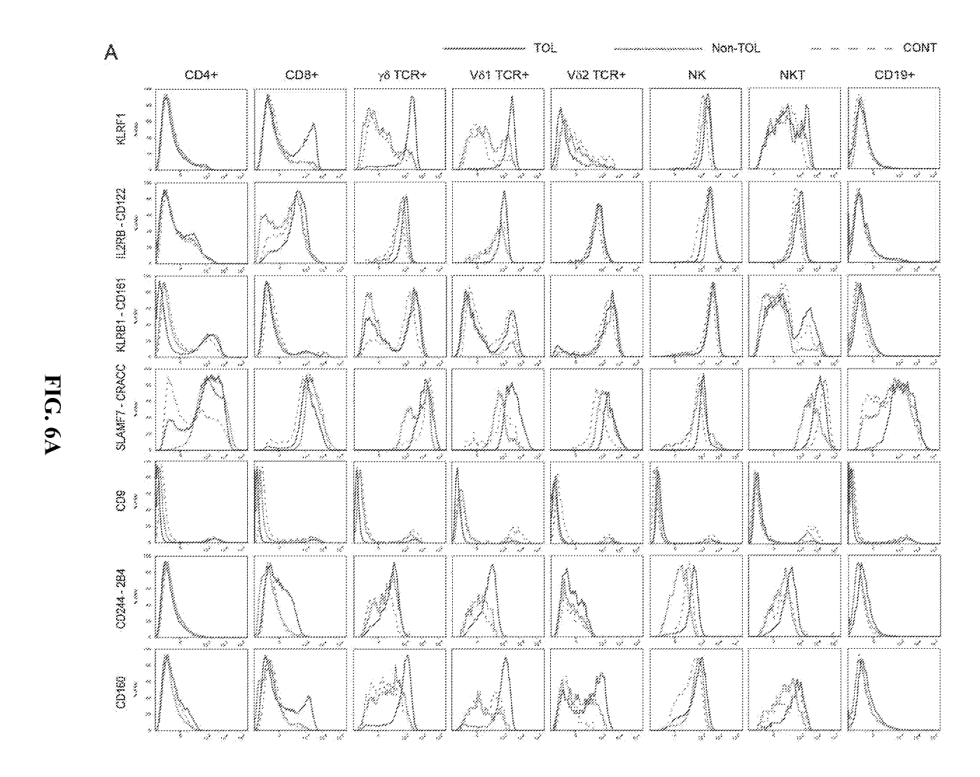

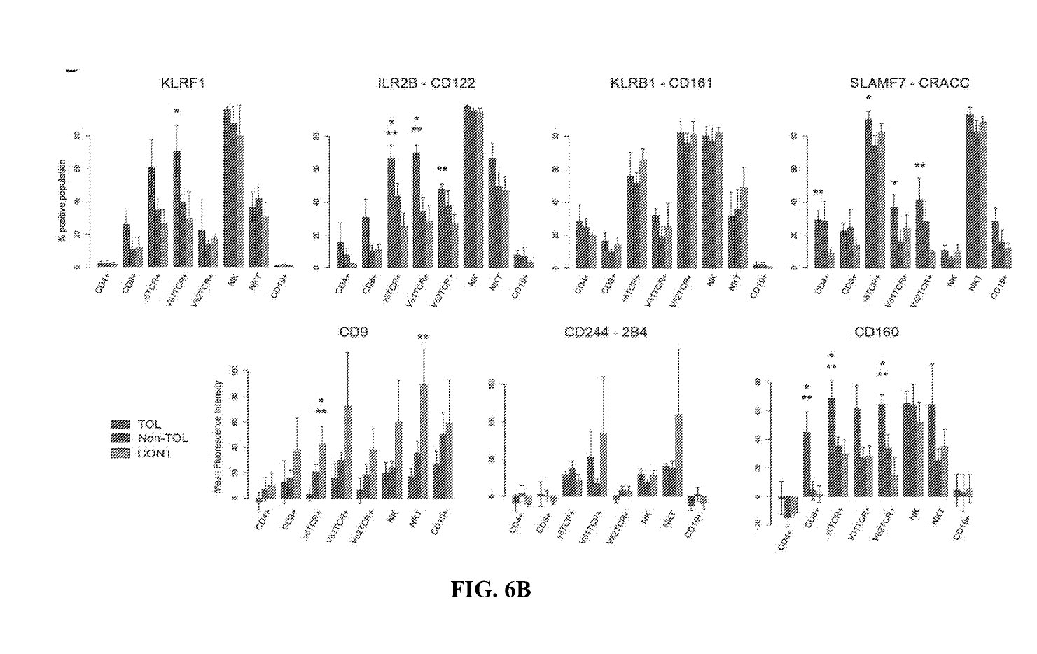

An initial comparative statistical analysis employing Significant Analysis of Microarrays (SAM) yielded a total of 2482 probes (corresponding to 1932 genes and 147 ESTs) with a false discovery rate (FDR) <5% (FIG. 2a). To identify the minimal set of genes capable of predicting the tolerant state, Predictive Analysis of Microarrays (PAM) analysis was performed in parallel on the same two groups of samples resulting in the identification of a subset of 26 probes, corresponding to 23 genes (all of them present in the SAM list; FIG. 2b), capable of correctly classifying tolerant recipients with an overall error rate of 0.026, (sensitivity 1, specificity 0.944). Multidimensional scaling analysis was then performed to visually represent the proximity between TOL and Non-TOL samples according to the expression of the 26 probes. As depicted in FIG. 2c, TOL and Non-TOL samples appeared as two clearly separated groups. Overall, analysis of microarray-derived expression data results in the identification of a genetic classifier that exhibits high accuracy at discriminating TOL from Non-TOL samples.

### Example 9

### Prediction of Tolerance in Stable Liver Recipients Under Maintenance Immunosuppression Employing Microarray Expression Data

To estimate the proportion of potentially tolerant individuals among stable liver recipients and thus externally validate the tolerance-related 26-probe microarray signature, corresponding to the 23-gene microarray signature, we employed PAM to classify a cohort of 19 STA patients into TOL and Non-TOL categories. Tolerance was predicted in 26% of cases (this rate ranged from 21 to 31% when three other prediction algorithms, namely supervector machine learning using the kernel radial basis function (SVM-rbf) or linear kernel (SVM-lin), and Knearest neighbors, were employed; data not shown). This estimation is concordant with the rate of successful weaning we have observed in similarly selected stable liver recipients (5, 8). Furthermore, STA recipients identified as tolerant based on microarray expression patterns exhibited a higher proportion of peripheral blood Vδ1TCR+ T cells and Vδ1/Vδ2 T cell ratio than those identified as non-tolerant recipients (FIG. 3a), which is in agreement with two previous immunophenotyping studies (2, 3). Multidimensional scaling was next employed to plot TOL, Non-TOL and STA samples together based on the PAM-derived microarray expression signature. Notably, STA samples were grouped together with TOL or Non-TOL samples in concordance with their predicted clinical phenotype (FIG. 3b).

### Example 10

### Validation of Microarray Expression Data by qPCR

We employed qPCR to confirm the expression of the target genes identified by microarrays and to compare the expression measurements obtained from liver recipients with those from non-transplanted healthy individuals (CONT). Selected target genes for qPCR experiments included the 23 genes selected by PAM, 44 genes selected among those most highly ranked in the SAM-derived gene list, and 6 genes (UBD, HLA-DOB, FOXP3, LTBP3, MAN1A1, LGALS3) derived from previously published reports (Table 2).

TABLE 2
Results of qPCR gene expression experiments.

| Gene | Fold change | Fold change | P-value | P-value | P < 0.05 | P < 0.05 |
|---|---|---|---|---|---|---|
| symbol | Tol vs Non-Tol | Cont vs Tol | Tol vs Non-Tol | Tol vs Cont | Tol vs Non-Tol | Tol vs Cont |
| CLIC3 | 2.189 | 1.141 | 4.151E−06 | 1.228E−01 | X | |
| KLRF1 | 1.879 | 1.288 | 6.755E−06 | 1.730E−02 | X | X |
| SLAMF7 | 1.414 | 1.181 | 1.381E−05 | 4.835E−02 | X | X |
| FEZ1 | 2.219 | 1.474 | 2.179E−05 | 6.350E−02 | X | X |
| CD160 | 2.078 | 1.693 | 2.635E−05 | 2.114E−02 | X | X |
| CTBP2 | 1.542 | 1.165 | 4.371E−05 | 2.199E−02 | X | X |
| IL2RB | 1.641 | 1.434 | 1.054E−04 | 2.704E−02 | X | X |
| OSBPL5 | 1.699 | 1.347 | 1.193E−04 | 3.469E−03 | X | X |
| NKG7 | 1.510 | 1.380 | 2.562E−04 | 3.280E−03 | X | X |
| FLJ14213 | 1.759 | −1.165 | 2.824E−04 | 6.278E−01 | X | |
| GNPTAB | 1.329 | 1.003 | 4.302E−04 | 3.170E−01 | X | |
| PTGDR | 1.564 | 1.185 | 7.148E−04 | 1.788E−01 | X | |
| FEM1C | −1.380 | −1.395 | 8.222E−04 | 1.657E−03 | X | X |
| ZNF295 | −1.879 | −1.053 | 1.063E−03 | 5.192E−01 | X | |
| KLRD1 | 1.521 | 1.231 | 1.092E−03 | 1.976E−01 | X | |
| RGS3 | 1.717 | 1.021 | 1.492E−03 | 6.282E−01 | X | |
| CX3CR1 | 1.741 | −1.161 | 1.981E−03 | 3.870E−01 | X | |
| PSMD14 | 1.157 | 1.042 | 2.670E−03 | 1.925E−01 | X | |
| WDR67 | 1.248 | −1.169 | 2.735E−03 | 1.388E−01 | X | |
| PTCH1 | 1.390 | 1.223 | 2.850E−03 | 1.428E−01 | X | |
| ERBB2 | 1.939 | 1.161 | 3.286E−03 | 6.274E−01 | X | |
| GEMIN7 | 1.270 | −1.102 | 3.662E−03 | 3.954E−01 | X | |
| CD9 | 1.223 | 1.261 | 4.225E−03 | 1.468E−02 | X | X |
| CD244 | 1.371 | 1.202 | 4.250E−03 | 9.183E−02 | X | |
| NCALD | 1.366 | 1.189 | 5.190E−03 | 6.604E−02 | X | |
| EPS8 | 1.434 | 1.366 | 5.615E−03 | 2.913E−02 | X | X |
| PDE4B | −1.521 | −1.007 | 7.337E−03 | 7.564E−01 | X | |
| KLRB1 | 1.292 | 1.032 | 7.491E−03 | 7.171E−01 | X | |
| ZNF267 | −1.542 | 1.185 | 8.269E−03 | 2.471E−03 | X | X |
| FANCG | 1.257 | −1.010 | 1.392E−02 | 1.203E−01 | X | |
| UBD | 1.753 | 1.532 | 3.070E−02 | 6.397E−02 | X | X |
| ALG8 | 1.177 | −1.129 | 3.095E−02 | 3.180E−01 | X | |
| MAN1A1 | 1.218 | 1.270 | 3.145E−02 | 3.242E−03 | X | X |
| IL8 | −4.579 | 1.682 | 3.661E−02 | 1.023E−02 | X | X |
| DCTN2 | 1.083 | 1.007 | 8.705E−02 | 8.754E−01 | | |
| DAB2 | 1.279 | 1.240 | 1.110E−01 | 1.550E−01 | | |
| FOXP3 | 1.310 | −1.072 | 1.218E−01 | 2.926E−01 | | |
| UBE2V2 | 1.072 | −1.094 | 1.315E−01 | 2.393E−01 | | |
| PPM1B | −1.253 | −1.061 | 1.344E−01 | 2.996E−01 | | |
| NOTCH2 | 1.110 | 1.149 | 1.439E−01 | 2.420E−02 | | X |
| DOCK11 | −1.057 | −1.050 | 1.605E−01 | 2.943E−01 | | |
| THBD | −1.261 | 1.141 | 1.654E−01 | 1.600E−01 | | |
| PPM1B | −1.106 | −1.087 | 1.737E−01 | 3.970E−01 | | |
| UCHL5 | 1.061 | −1.061 | 1.840E−01 | 7.136E−01 | | |
| NOLA1 | 1.352 | −1.653 | 1.988E−01 | 1.273E−06 | | X |
| PSMF1 | 1.279 | 1.017 | 2.131E−01 | 3.000E−01 | | |
| TGFBR3 | 1.091 | 1.218 | 2.157E−01 | 8.922E−02 | | |
| C10orf119 | 1.193 | −1.007 | 2.244E−01 | 5.148E−01 | | |
| DCUN1D1 | 1.003 | −1.057 | 3.003E−01 | 7.313E−01 | | |
| HIP2 | 1.017 | −1.042 | 3.046E−01 | 8.832E−01 | | |
| RAD23B | −1.007 | 1.079 | 3.147E−01 | 2.379E−01 | | |
| TRIAP1 | −1.007 | −1.068 | 3.286E−01 | 2.516E−01 | | |
| EIF5A | −1.064 | 1.102 | 4.298E−01 | 3.466E−02 | | X |
| TRD@ | 1.075 | −1.297 | 4.494E−01 | 1.622E−01 | | |
| LTBP3 | −1.117 | −1.390 | 4.685E−01 | 6.387E−03 | | X |
| HLA-DOB | −1.133 | −1.165 | 5.054E−01 | 2.698E−01 | | |
| RB1CC1 | −1.028 | −1.214 | 5.303E−01 | 2.965E−03 | | X |
| ATXN10 | −1.025 | −1.169 | 5.549E−01 | 1.649E−03 | | X |
| TRA@ | −1.173 | −2.078 | 5.959E−01 | 9.081E−04 | | X |
| MRPS31 | 1.261 | −1.429 | 6.005E−01 | 6.246E−05 | | X |
| IKZF3 | 1.031 | −1.16 | 6.317E−01 | 1.080E−01 | | |
| DTNBP1 | 1.193 | 1.075 | 6.541E−01 | 6.375E−01 | | |
| GRSF1 | −1.032 | −1.157 | 6.813E−01 | 3.847E−02 | | X |
| UBB | 1.091 | 1.025 | 7.206E−01 | 1.044E−01 | | |
| NOLA1 | −1.014 | −1.165 | 7.708E−01 | 1.147E−02 | | X |
| C10orf110 | 1.376 | 1.149 | 7.996E−01 | 8.534E−01 | | |
| COPZ1 | −1.053 | −1.053 | 8.605E−01 | 5.216E−01 | | |
| LGALS3 | −1.003 | 1.270 | 8.927E−01 | 2.077E−02 | | X |
| S100A10 | −1.025 | −1.068 | 9.557E−01 | 7.348E−01 | | |

Peripheral blood samples from 16 TOL, 15 Non-TOL and 16 CONT individuals were employed for these experiments. TOL and Non-TOL samples differed in the expression of 34 genes (Table 2 and FIG. 4a). Thirty genes were differentially expressed when assessed by microarrays but not by qPCR. Among these, PCR primers and microarray probes did not recognize the same transcripts in 11 cases. Hence, qPCR could confirm the differential expression of 64% of the genes selected by microarrrays. The reproducibility of qPCR expression values was assessed by computing inter-patient and inter-assay variation. Inter-patient variation (median SD of ΔC T=0.68) greatly exceeded inter-assay variation (median SD of ΔC T=0.21). This suggests that the variability of the qPCR is small enough to reliably detect differences in gene expression between TOL and Non-TOL recipients. Although target genes had been selected on account of their differential expression between TOL and Non-TOL samples, there were 26 genes differentially expressed between TOL and CONT samples as well (Table 2 and FIG. 4a). The similarities between TOL, Non-TOL and CONT expression patterns were then assessed in an unsupervised manner through multidimensional scaling analysis. This resulted in CONT samples being clustered in between TOL and Non-TOL groups (FIG. 4b). Taken together, qPCR expression results confirm the validity of most genes identified by microarrays and reveal that tolerance-related expression patterns differ from both Non-TOL recipients and non-transplanted healthy individuals, albeit TOL recipients appear to be closer to healthy individuals than to Non-TOL recipients.

### Example 11

### Prediction of Tolerance in an Independent Validation Test Employing qPCR-Derived Gene Models

Among the candidate biomarkers identified in qPCR experiments on the basis of their differential expression between TOL and Non-TOL samples, we searched for those that would form optimal parsimonious models capable of predicting tolerance status in an independent validation set. This was accomplished by utilizing a novel classification modelling approach based on the Misclassified Penalized Posterior (MiPP) algorithm and incorporating an independent cohort of 11 TOL and 12 Non-TOL recipients not previously employed for data analysis and from whom no microarray data were available. MiPP selected 3 signatures of 2, 6, and 7 genes (altogether comprising 12 different genes) capable of correctly classifying samples included in both the training and validation sets (Table 3).

TABLE 3
Most predictive genetic classifiers identified by
MiPP in qPCR expression data set and their
performance in training and independent test sets ^{A}.

| | Selec- | Pre- | Class | Mean ER | Mean ER |
|---|---|---|---|---|---|
| | tion | diction | com- | in train- | in vali- |
| Gene signatures | method | rule | parison | ing set | dation set |
| KLRF1, SLAMF7 | MiPP | LDA, | 2-class | 0.064 | 0.13 |
| | | QDA, | | | |
| | | SVM-rbf | | | |
| KLRF1, NKG7, | MiPP | SVM-rbf | 2-class | 0.032 | 0.17 |
| IL2RB, KLRB1, | | | | | |
| FANCG, GNPTAB | | | | | |
| SLAMF7, KLRF1, | MiPP | SVM-lin | 2-class | 0.064 | 0.13 |
| CLIC3, PSMD14, | | | | | |
| ALG8, CX3CR1, | | | | | |
| RGS3 | | | | | |

^{A} Abbreviations:
ER: overall error rate;
MiPP: misclassified posterior probability;
LDA: Lineal discriminant analysis;
QDA: quadratic discriminant analysis;
SVM-rbf: supervector machine with radial basis function;
SVM-lin: supervector machine with lineal function as kernel.

These experiments indicate that qPCR can be employed on peripheral blood samples to derive robust, reproducible and highly accurate gene models of liver operational tolerance.

### Example 12

### Identification of Clinical Variables Implicated in the Tolerance-Associated Gene Signature

We performed Globaltest to assess the influence of age, gender, type of immunosuppression, time from transplantation, peripheral blood leukocyte counts, and hepatitis C virus (HCV) infection status on peripheral blood microarray gene expression patterns. No significant correlation was found between the tolerance-related expression profile and patient age, gender, pharmacological immunosuppression and peripheral blood lymphocyte, neutrophil and monocyte numbers (data not shown). Time from transplantation was marginally associated with the PAM-derived 26-probe signature corresponding to 23 distinct genes (Pvalue <0.042) but not with the 2462-probe set identified by SAM. HCV infection, in contrast, had a major impact both on global gene expression patterns and on the tolerance-related expression signatures (P-value <0.0003 and 0.0033 for the 26-and the 2462-probe sets, respectively). To further dissect the effects of HCV infection on gene expression patterns following transplantation we compared samples from chronically infected patients (HCV-pos) with those of non-infected (HCV-neg) recipients employing SAM. This resulted in the identification of 4725 differentially expressed probes (FDR<5%; data not shown). Further, we used SAM to compare TOL and NonTOL samples stratified on the basis of HCV infection status. HCV-neg TOL and Non-TOL individuals differed in 117 probes, while 528 probes were differentially expressed between HCV-pos TOL and Non-TOL recipients (FDR<5%; FIG. 5a). HCV infection was also found to influence the expression of 12 out of the 26 probes included in the PAM-derived microarray genetic classifier, albeit correlation was tighter with tolerance than with HCV infection (FIG. 5b). This is concordant with our finding that the 26 probe set classifies TOL and Non-TOL samples regardless of HCV infection status (FIG. 5c). Thus, while HCV infection has a major influence on peripheral blood gene expression following liver transplantation, this does not prevent accurate discrimination between TOL and Non-TOL recipients.

### Example 13

### PBMC Subsets Involved in the Tolerance-Related Gene Expression Footprint

In a previous report (3) we investigated in detail the differences in PBMC subsets between TOL and Non-TOL liver recipients (this report included 32 out of the 38 TOL and Non-TOL recipients incorporated in our current microarray study). TOL recipients exhibited an increased number of CD4+CD25+Foxp3+, γδTCR+ and δ1TCR+ T cells. In contrast, no differences were observed in the frequency or absolute numbers of other T cell subsets, B, NK and NKT cells (3). To determine the contribution of these PBMC subsets to tolerance-associated expression patterns we employed Globaltest to correlate cell subset frequencies with microarray-derived expression levels. All 57 patients from whom microarray data were available (including TOL, Non-TOL and STA recipients) were employed for this study. First we computed the number of probes from the SAM-derived 2482-probe list whose expression correlated with the frequency of each specific PBMC subset. NK, Vδ1TCR+ and total γδTCR+ T cells influenced 314, 296 and 438 probes, respectively, although statistical significance was only reached for NK (P-value <0.0032) and γδTCR+ T cells (P-value <0.0271). For comparison, a similar analysis was then conducted on the 4725-probe list differentiating HCV-pos from HCV-neg samples. This analysis identified CD8+ T cells as the lymphocyte subset influencing the greatest number of genes, although this did not reach statistical significance (328 probes, P<0.14). NK, γδTCR+ and Vδ1TCR+ peripheral blood lymphocyte proportions also correlated with the expression of multiple individual genes included in the PAM-derived 26-probe set (FIG. 5c), although only γδTCR+ T cell frequency was shown to be significantly associated with the 26-probe set as a whole (P-value <0.0154). The results of these analyses indicate that both NK and γδTCR+ T cells influence tolerance-associated peripheral blood expression patterns. Considering that TOL and Non-TOL recipients differ in the number of peripheral blood γδTCR+ T cells (3), it is clear that tolerance-related differential gene expression can be attributed, at least in part, to an increased number of γδTCR+ T cells in TOL recipients. Regarding NK cells, which are present in similar numbers in TOL and Non-TOL recipients, we hypothesized that the significant correlation observed might be due to changes in their transcriptional program. To test this hypothesis and further assess the contribution of other PBMC subsets, we measured by flow cytometry the protein levels of IL2RB, KLRB1, CD244, CD9, KLRF1, CD160 and SLAMF7 on CD4+, CD8+, γδTCR+ T cells, NK, CD19+ and NKT cells from 6 TOL, 6 Non-TOL and 5 healthy individuals. These proteins were mainly expressed on NK, NKT and γδTCR+ T cells, with significant differences being noted between TOL, Non-TOL and CONT individuals (FIGS. 6a and b). These findings indicate that TOL and Non-TOL recipients differ in the expression program of several PBMC subsets, mainly Vδ1TCR+ T cells and NK cells, and that in many cases these expression changes are unique to the tolerant state.

### Example 14

### Peripheral Blood Immunophenotyping on Sorted PBMC Subsets

The expression at the protein level of 7 of the most discriminative genes identified by microarray and qPCR experiments (ILRB2, KLRB1, CD244, CD9, KLRF1, CD160, SLAMF7) was assessed on sorted PBMC subpopulations from a subset of 6 TOL, 6 Non-TOL and 5 CONT patients. CD160 fluorescent monoclonal antibodies were purchased from Beckman Coulter, SLAMF7 and KLRF1 from R&D Systems. All remaining antibodies were purchased from BD Biosciences.

### REFERENCES

1. Lerut, J., and Sanchez-Fueyo, A. 2006. An appraisal of tolerance in liver transplantation. Am J Transplant 6:1774-1780.
2. Li, Y., Koshiba, T., Yoshizawa, A., Yonekawa, Y., Masuda, K., Ito, A., Ueda, M., Mori, T., Kawamoto, H., Tanaka, Y., et al. 2004. Analyses of peripheral blood mononuclear cells in operational tolerance after pediatric living donor liver transplantation. Am J Transplant 4:2118-2125.
3. Martinez-Llordella, M., Puig-Pey, I., Orlando, G., Ramoni, M., Tisone, G., Lerut, J., Latinne, D., Rimola, A., Brouard, S., Soulillou, J., et al. 2007. Multiparameter of immune profiling of operational tolerance in liver transplantation. Am J Transplant 7:309-319.
4. Mazariegos, G. V., Zahorchak, A. F., Reyes, J., Ostrowski, L., Flynn, B., Zeevi, A., and Thomson, A. W. 2003. Dendritic cell subset ratio in peripheral blood correlates with successful withdrawal of immunosuppression in liver transplant patients. Am J Transplant 3:689-696.
5. Brouard, S., Mansfield, E., Braud, C., Li, L., Giral, M., Hsieh, S. C., Baeten, D., Zhang, M., Ashton-Chess, J., Braudeau, C., et al. 2007. Identification of a peripheral blood transcriptional biomarker panel associated with operational renal allograft tolerance. Proc Natl Acad Sci USA 104:15448-15453.
6. Alizadeh, A., Eisen, M., Davis, R. E., Ma, C., Sabet, H., Tran, T., Powell, J. I., Yang, L., Marti, G. E., Moore, D. T., et al. 1999. The lymphochip: a specialized cDNA microarray for the genomic-scale analysis of gene expression in normal and malignant lymphocytes. Cold Spring Harb Symp Quant Biol 64:71-78.
7. Bussey, K. J., Kane, D., Sunshine, M., Narasimhan, S., Nishizuka, S., Reinhold, W. C., Zeeberg, B., Ajay, W., and Weinstein, J. N. 2003. MatchMiner: a tool for batch navigation among gene and gene product identifiers. Genome Biol 4:R27.
8. Louis, S., Braudeau, C., Giral, M., Dupont, A., Moizant, F., Robillard, N., Moreau, A., Soulillou, J. P., and Brouard, S. 2006. Contrasting CD25hiCD4+T cells/FOXP3 patterns in chronic rejection and operational drug-free tolerance. Transplantation 81:398-407.
9. Hernandez-Fuentes, M., Sawitzki, B., Sagoo, P., Craciun, L., Brouard, S., Perucha, E., Chapman, S., Bradeau, C., Peters, B., Roberts, I., et al. 2007. Biomarkers of tolerance in kidney transplants. Am J Transplant 7:340 (Abst 734).
10. Wu, Z., Irizarri, R. A., Gentleman, R., Murillo, F. M., and Spencer, F. 2004. A Model Based Background Adjustment for Oligonucleotide Expression Arrays. JASA 99:909-917.
11. Johnson, W. E., Li, C., and Rabinovic, A. 2007. Adjusting batch effects in microarray expression data using empirical Bayes methods. Biostatistics 8:118 127.
12. Tusher, V. G., Tibshirani, R., and Chu, G. 2001. Significance analysis of microarrays applied to the ionizing radiation response. Proc Natl Acad Sci USA 98:5116-5121.
13. Tibshirani, R., Hastie, T., Narasimhan, B., and Chu, G. 2002. Diagnosis of multiple cancer types by shrunken centroids of gene expression. Proc Natl Acad Sci USA 99:6567-6572.
14. Goeman, J. J., van de Geer, S. A., de Kort, F., and van Houwelingen, H. C. 2004. A global test for groups of genes: testing association with a clinical outcome. Bioinformatics 20:93-99.
15. Sawitzki, B., Bushell, A., Steger, U., Jones, N., Risch, K., Siepert, A., Lehmann, M., Schmitt-Knosalla, I., Vogt, K., Gebuhr, I., et al. 2007. Identification of gene markers for the prediction of allograft rejection or permanent acceptance. Am J Transplant 7:1091-1102.
16. Ocklenburg, F., Moharregh-Khiabani, D., Geffers, R., Janke, V., Pfoertner, S., Garritsen, H., Groebe, L., Klempnauer, J., Dittmar, K. E., Weiss, S., et al. 2006. UBD, a downstream element of FOXP3, allows the identification of LGALS3, a new marker of human regulatory T cells. Lab Invest 86:724-737.
17. Soukup, M., Cho, H., and Lee, J. K. 2005. Robust classification modeling on microarray data using misclassification penalized posterior. Bioinformatics 21 Suppl 1:i423-430.
18. Su, A. I., Wiltshire, T., Batalov, S., Lapp, H., Ching, K. A., Block, D., Zhang, J., Soden, R., Hayakawa, M., Kreiman, G., et al. 2004. A gene atlas of the mouse and human protein-encoding transcriptomes. Proc Natl Acad Sci USA 101:6062-6067.
19. N. Najafian et al., “How can we measure immunologic tolerance in humans?” J. Am. Soc. Nephrol. 2006, vol. 17, pp. 2652-63;
20. K. A. Newell et al., “Tolerante assays: measuring the unknown”, Transplantation 2006, vol. 81, pp. 1503-9;
21. J. Cai et al., “Minor H antigen HA-1-specific regulator and effector CD8+ T cells, and HA-1 microchimerism, in allograft tolerance”, J. Exp. Med. 2004, vol. 199, pp. 1017-23;
22. E. Jankowska-Gan E et al., “Human liver allograft acceptance and the “tolerance assay”, Hum. Immunol. 2002, vol. 63, pp. 862-70;
23. M. Hernandez-Fuentes et al., “Indices of tolerance: Interim report”, abstract #999, World Transplant Congress 2006;
24. WO 02/084567
25. S. Brouard et al., “Operationally tolerant and minimally immunosuppressed kidney recipients display strongly altered blood T-cell clonal regulation”, Am. J. Transplant. 2005, vol. 5, pp. 330-40;
26. WO 2008/081039
27. Compton, J. (1991) Nature, 350, 91-92
28. Guatelli, J. C., Whitfield, K. M., Kwoh, D. Y., Barringer, K. J., Richman, D. D. and Gingeras, T. R. (1990) Proc. Natl Acad. Sci. USA, 87, 1874-1878
29. Notomi et al. Nucleic Acids Research, 2000, Vol. 28, No. 12 E63-e63

1.-18. (canceled)

19. Method for in vitro diagnosis/prognosis of the tolerant state of a patient which, has been or is going to be, respectively, subject of a liver transplantation, comprising the steps of:
a) obtaining a biological sample from the patient and
b) measuring the expression levels in that sample of a group of genes selected among: KLRF1 (NCBI51348) and SLAMF7 (NCBI57823); or KLRF1 (NCBI51348), NKG7 (NCBI4818), IL2RB (NCBI3560), KLRB1 (NCBI3820), FANCG (NCBI2189) and GNPTAB (NCBI79158); or SLAMF7 (NCBI57823), KLRF1 (NCBI51348), CLIC3 (NCBI9022), PSMD 14 (NCBI10213), ALG8 (NCBI79053), CX3CR1 (NCBI1 524) and RGS3 (NCBI5998) and
c) comparing the expression fingerprint of each group of genes with the expression levels of the same group of genes of a control biological sample obtained from a non-tolerant liver transplant recipient requiring on-going immunosuppression therapy and
d) having instructions to assess tolerance or non-tolerance to liver transplantation of the patient whose biological sample has been assayed, based on the up-regulation of the expression of any of said group of genes with regard to expression threshold values for each gene of the same group of genes of the control sample.

20. Method according to claim 19 wherein the biological sample is a blood sample.

21. Method according to claim 20 wherein gene expression levels are measured specifically in Vδ1TCR+ blood cell subtype.

22. Method according to claim 19 wherein gene expression levels are measured for the group of genes consisting in KLRF1 (NCBI51348) and SLAMF7 (NCBI57823).

23. Method according to claim 22 wherein additionally the gene expression levels of any of the following genes is measured: IL2RB (NCBI3560), KLRB1 (NCBI3820), CD9 (NCBI928), CD244 (NCBI51744) or CD160 (NCBI11126).

24. Method according to claim 19, wherein the measurement of the gene expression levels is carried out using a microarray or a gene chip which comprises nucleic acid probes, said nucleic acid probes comprising sequences that specifically hybridize to the transcripts of the corresponding set of genes.

25. Method according to claim 24, wherein the microarray is a cDNA microarray or an oligonucleotide microarray.

26. Method according to claim 19, wherein the measurement of the gene expression levels is carried out by quantitative reverse transcription polymerase chain reaction of RNA extracted from the sample or by isothermal amplification.

27. Method according to claim 19, wherein measuring the gene expression levels is carried out by detecting the proteins encoded by the corresponding genes.

28. Method according to claim 27, wherein the proteins are detected by antibodies specific to said proteins, by a proteins chip or by HPLC.

29. Method according to claim 27 wherein the proteins are detected in the specific blood cell subtype Vδ1TCR+.

30. Method according to claim 19, for the diagnosis of the tolerant state of a patient which has been the subject of a liver transplantation.

31. Method according to claim 19, wherein the biological sample is a blood sample, gene expression levels are measured specifically in Vδ1TCR+ blood cell subtype, and the measurement of the gene expression levels is carried out using a microarray or a gene chip which comprises nucleic acid probes, said nucleic acid probes comprising sequences that specifically hybridize to the transcripts of the corresponding set of genes.

32. Method according to claim 19, wherein the biological sample is a blood sample, gene expression levels are measured specifically in Vδ1TCR+ blood cell subtype, and the measurement of the gene expression levels is carried out by quantitative reverse transcription polymerase chain reaction of RNA extracted from the sample or by isothermal amplification.

33. Kit for performing the method of claim 19, comprising (i) means for measuring the gene expression levels of the corresponding group of genes, wherein said means permit to measure the expression of no more than 500 distinct genes; and (ii) instructions for correlating the gene expression levels above or below pre-determined threshold values indicative of the tolerant state in liver transplantation.

34. Kit according to claim 33, wherein the means comprise a microarray or a gene chip which comprises nucleic acid probes, said nucleic acid probes comprising sequences that specifically hybridize to the transcripts of the corresponding set of genes, wherein said nucleic microarray comprises nucleic acid probes comprising sequences that specifically hybridize to no more than 500 distinct genes.

35. Kit according to claim 34, further comprising reagents for performing a microarray analysis.

36. Kit according to claim 33, wherein the means comprise oligonucleotide primers for performing a quantitative reverse transcription polymerase chain reaction, said primers comprising sequences that specifically hybridize to the complementary DNA derived from the transcripts of the corresponding set of genes, and wherein said primers comprise sequences that specifically hybridize to no more than 500 distinct genes.

37. Kit according to claim 34 comprising a solid support wherein nucleic acid probes which comprises sequences that specifically hybridize to the transcripts of the corresponding set of genes are displayed thereon.

38. A method for selecting or modifying a immunotherapy treatment protocol, either before of after liver transplantation was performed, by assessing the tolerant state of the liver recipient by using the method of claim 19.

39. A method for selecting or modifying a immunotherapy treatment protocol, either before of after liver transplantation was performed, by assessing the tolerant state of the liver recipient by using the kit of claim 33.
